(19) United States
(12) Patent Application Publication
(10) Pub. No.: US 2014/0277191 A1
(43) Pub. Date: Sep. 18, 2014

(54) ARTHRODESIS DEVICE AND METHOD OF USE

(71) Applicant: THE CLEVELAND CLINIC FOUNDATION, Cleveland, OH (US)

(72) Inventors: Peter J. Evans, Shaker Hts., OH (US); Bret E. Hartzell, Canal Fulton, OH (US)

(73) Assignee: THE CLEVELAND CLINIC FOUNDATION, Cleveland, OH (US)

(21) Appl. No.: 14/203,605

(22) Filed: Mar. 11, 2014

Related U.S. Application Data
(60) Provisional application No. 61/777,128, filed on Mar. 12, 2013.

(51) Int. Cl. A61B 17/72

(52) U.S. Cl. CPC A61B 17/7225
USPC 606/308; 606/316

(57) ABSTRACT

A fastening device for compressing first and second bones together includes a first member configured to extend entirely through the first bone and having a shaft that includes a distal threaded portion and an unthreaded portion. The threaded portion has an outer diameter that is not larger than the diameter of the unthreaded portion. A second member has a first threaded portion for securing to the second bone and a second threaded portion for engaging the threaded portion of the first member while the first member extends entirely through the first bone to secure the first and second members together and compress the first and second bones.

### RELATED APPLICATIONS

This application claims priority to U.S. Provisional Application No. 61/777,128, filed Mar. 12, 2013, the subject matter of which is incorporated herein in its entirety.

### TECHNICAL FIELD

The present invention relates to a fastener for securing multiple bones together and, in particular, relates to a joint arthrodesis compression device.

### BACKGROUND

In the field of orthopedics, joints between bones in the hand and feet may become altered due to injury, disease, etc. to the point that joint functionality is impaired or lost. As a result, it may be necessary to fuse the bones forming the joint to help alleviate pain or help prevent damage to surrounding soft tissue. In one example, the distal phalanx and middle phalanx are fused at the distal interphalangeal (DIP) joint. Currently, fusion is achieved using dual threaded screws, K-wires, and compression screws. These devices, however, are flawed in that they cannot alter the bone compression level in vivo. These devices are also unable to achieve solid bone fixation in the middle phalanx.

### SUMMARY OF THE INVENTION

In accordance with the present invention a fastening device for compressing first and second bones together includes a first member configured to extend entirely through the first bone and having a shaft that includes a threaded portion and an unthreaded portion. The threaded portion has an outer diameter that is not larger than the diameter of the unthreaded portion. A second member has a first threaded portion for securing to the second bone and a second threaded portion for engaging the threaded portion of the first member while the first member extends entirely through the first bone to secure the first and second members together and compress the first and second bones.

In accordance with another aspect of the present invention a fastening device for compressing first and second bones together includes a first member having a head and a shaft extending from the head. The head has a first cooperating member and the shaft includes threads for engaging the first bone. A second member has a head and a shaft extending from the head. The head has a second cooperating member that is engageable with the first cooperating member of the first member to compress the first and second bones. The shaft includes threads for engaging the second bone.

In accordance with another aspect of the present invention, a method of compressing first and second bones together includes providing a first member having a shaft that includes a threaded portion and an unthreaded portion, the threaded portion having an outer diameter that is not larger than an outer diameter of the unthreaded portion. A second member includes a first threaded portion and a second threaded portion. The first threaded portion of the second member is threaded into the second bone to secure the second member to the second bone. A passage is formed through the entire first bone having a diameter that is greater than the diameters of the shaft of the first member. The first member is inserted through the passage such that the shaft of the first member extends entirely though the first bone. The threaded portion of the first member is threaded into the second threaded portion of the second member to compress the first and second bones together.

In accordance with another aspect of the present invention, a method of compressing first and second bones together includes providing a first member having a head and a shaft extending from the head, the head having a first cooperating member and the shaft including threads for engaging the first bone. A second member includes a head and a shaft that extends from the head, the shaft including threads for engaging the second bone and the head has a second cooperating member that is releasably engageable with the first cooperating member of the first member. The threads of the first member are thread into the first bone to secure the first member to the first bone. The first threaded portion of the second member is threaded into the second bone to secure the second member to the second bone. The cooperating members of the first and second members are secured together to compress the first and second bones together.

### BRIEF DESCRIPTION OF THE DRAWINGS

Other objects and advantages and a fuller understanding of the invention will be had from the following detailed description of the preferred embodiments and the accompanying drawings, wherein:

FIG. 1 is a schematic illustration of a bone fastening device in accordance with an aspect of the present invention.

FIG. 2 is an exploded assembly view of the bone fastening device of FIG. 1.

FIG. 3 is a cross-sectional view of a component of the device of FIG. 1 taken along line 3-3 of FIG. 2.

FIGS. 4A-4C are schematic illustrations of the bone fastening device of FIG. 1 being implanted in first and second bones.

FIG. 5 is a schematic illustration of the bone fastening device of FIG. 1 modified to secure three bones together.

FIG. 6 is a schematic illustration of an alternative configuration for a component of the bone fastening device of FIG. 1.

FIG. 7 is a schematic illustration of an anteroposterior view of a bone fastening device in accordance with another aspect of the present invention.

FIG. 8 is an exploded assembly view of the bone fastening device of FIG. 7.

FIG. 9 is a schematic illustration of a lateral view of a bone fastening device in accordance with another aspect of the present invention.

FIG. 10A is an enlarged side view of an alternative connection between components of the bone fastening device of FIG. 7.

FIG. 10B is a side view of an alternative connection between components of the bone fastening device of FIG. 7.

FIG. 10C is a side view of another alternative connection between components of the bone fastening device of FIG. 7.

### DETAILED DESCRIPTION

The present invention relates to a fastener for securing multiple bones together and, in particular, relates to a joint arthrodesis compression device. The device of the present invention may be used to help secure and/or fuse together any bones associated with a joint, e.g., spinal facet joints, mandibular joints, carpal bones, metacarpal bones, phalanges of the hand, tarsal bones, metatarsal bones, and phalanges of the foot. The device of the present invention may also be used in connection with joint arthroplasty devices, e.g., used with articulating components, and, thus, may be sized for implantation within long bones of the body, e.g., tibia, fibula, radius, ulna, etc. As illustrated in the figures, the device is used to fuse together the distal and middle phalanges 120, 140 associated with the DIP joint 160 in the hand 150.

FIGS. 1-4C illustrate a fastening device 20 in accordance with an aspect of the present invention. The fastening device 20 is implanted in adjacent bones 120, 140 in the hand 150 that collectively define a joint 160. The fastening device 20 includes a first member 40 and a second member 80 releasably connected to the first member. The first and second members 40, 80 are formed from a durable, biocompatible material, e.g., titanium. The first member 40 has an elongated shape and extends along an axis 42 from a first end 44 to a second end 46. The first member 40 includes a head 50 having a generally hemispherical shape and defining a surface 54 that faces the second end 46 of the first member. As shown, the surface 54 is tapered although other shapes such as planar, curved or frustoconical are contemplated. The head 50 includes receiving means 52 configured to receive a tool, e.g., a screwdriver or Allen wrench, to facilitate driving the first member 40 into bone.

A shaft 60 extends from the head 50 along the axis 42 to the second end 46 of the first member 40. The shaft 60 has a circular cross-section and a length along the axis 42 indicated by L₁. The shaft 60 includes an unthreaded portion 62 and a threaded portion 64. The unthreaded portion 62 has an outer diameter φ₁ and is positioned axially between the threaded portion 64 and the head 50. The threaded portion 64 includes a series of threads 66 having a major or outer diameter of φ₂. The threads 66 may be helical, square, etc. In one example, the thread diameter φ₂ and the diameter φ₁ of the unthreaded portion 62 are the same. The thread diameter φ₂ may, however, be smaller than the diameter φ₁ of the unthreaded portion 62. In any case, the diameter φ₂ of the threads 66 is not greater than the diameter φ₁ of the unthreaded portion 62. The diameters φ₁, φ₂ may be on the order of about 1.5 mm to about 2 mm, although smaller or larger sizes are contemplated to accommodate the particular application anatomy.

The second member 80 extends along an axis 82 from a first end 84 to a second end 86. The second member 80 has a circular cross-section and a length along the axis 82 indicated by L₂. The second member 80 includes an outer surface 88 from which a series of radially extending, self-tapping threads 90 extend. The outer surface 88 tapers inwardly as the second member 80 extends from the first end 84 towards the second end 86. Alternatively, the outer surface 88 may have a substantially cylindrical shape (not shown). The threads 90 extend along the entire length L₂ of the second member 80 but may alternatively extend along only a portion or discrete portions of the length (not shown). The threads 90 have a major or outer diameter indicated at φ₃ and may be helical, square, etc. The diameter φ₃ of the threads 90 varies along the length L₂ of the second member 80 but may alternatively be constant along the entire length (not shown). In one example, the diameter φ₃ of the threads 90 decreases in a direction extending towards the second end 86 of the second member 80. The diameter φ₃ may be, for example, about 3.5 mm to about 5.5 mm.

The first end 84 of the second member 80 terminates at an axial end surface 100. The end surface 100 may be planar or may be contoured to mimic the shape of the surrounding bone in which the second member 80 is secured. In any case, a first passage 102 extends from the end surface 100 towards the second end 86 of the second member 80. The first passage 102 defines receiving means 104 having a hex or hexalobe shape configured to receive a tool, e.g., a hex driver or Allen wrench, to facilitate driving the second member 80 into bone. Referring to FIG. 3, a second passage 106 extends from the first passage 102 towards the second end 86 of the second member 80. The second passage 106 includes threads 108 that mate with the threads 66 on the first member 40 to secure the first and second members 40, 80 together. Consequently, the threads 66, 108 can have any corresponding size and shape so long as they are capable of interconnecting with one another.

Referring to FIGS. 4A-4C, the first and second bones 120, 140 secured together by the fixation device 20 of the present invention constitute the distal phalanx and middle phalanx of a finger on the hand 150 (see also FIG. 1). Skin and tissue 170 surround the bones 120, 140, and all are illustrated in phantom in FIGS. 4A-4C for purposes of clarity. The first bone 120 extends from a proximal end 122 to a distal end 130. The proximal end 120 terminates at an axial end surface 124 and the distal end 130 terminates at an axial end surface 132. The second bone 140 extends from a proximal end 142 to a distal end 144. The proximal end 142 terminates adjacent a third bone 180 (see FIG. 1), e.g., the proximal phalanx of the hand 150, that cooperates with the second bone 140 to form a joint 190, e.g., the proximal interphalangeal (PIP) joint. The distal end 144 terminates at an axial end surface 146 adjacent the axial end surface 124 of the first bone 120.

During the procedure, the first bone 120 and second bone 140 are prepared to receive the fastening device 20. Referring to FIG. 4A, a short, transverse incision, e.g., fish-mouth, is made in the distal end of the finger to be treated and spread using a small clip or snap (not shown). A K-wire (not shown) is advanced through the finger tissue at the joint 160 and passed antegrade through the first bone 120 to the distal end 130 of the first bone. The appropriate cannulated drill, shown schematically at 135, is slid over the K-wire and advanced retrograde entirely through the first bone 120 in the direction indicated generally by the arrow A, forming a passage 134 there through that terminates at the joint 160 between the bones 120, 140. In other words, the passage 134 in the first bone 120 therefore extends the entire length of the first bone between the end surfaces 124, 132. The diameter of the passage 134 is larger than the diameter(s) φ_{1, 2} of the shaft of the first member 80.

The joint 160 may then be reduced or resected as is known in the art. More specifically, due to disease, anatomical conditions or other factors, it may be desirable to reduce or resect a portion of the proximal end 122 of the first bone 120 and/or the distal end 144 of the second bone 140 prior to fusing the first and second bones together. Resection of the first and/or second bones 120, 140 can be performed using conventional techniques, with one exemplary bone resection illustrated in phantom in FIG. 4A. The end surface 124 of the proximal end 122 of the first bone 120 may therefore constitute the natural articulating surface of the first bone or may constitute the now exposed end surface of the first bone following resection, illustrated in phantom at 124′. Likewise, the end surface 146 of the distal end 144 of the second bone 140 may therefore constitute the natural articulating surface of the second bone or may constitute the now exposed end surface of the second bone following resection, illustrated in phantom at 146′.

In any case, the drill 135 is then advanced across the joint 160 into the second bone 140 to form a passage 148 in the second bone. The passage 148 extends from the end surface 146 towards the proximal end 142 of the second bone 140. The passage 148, however, does not extend entirely through the second bone 140 but extends at least as long as the length L₂ of the second member 80. The diameter of the passage 148 is smaller than the diameter φ₃ of the threads 108 of the second member 80. As illustrated in FIG. 4A, the passages 134, 148 are substantially co-axial with one another, i.e., the passages collectively extend in a straight line through the bones 120, 140. Alternatively, the passages 134, 148 may be angled relative to one another (not shown) based on anatomical conditions and/or design considerations.

Referring to FIG. 4B, the second end 86 of the second member 80 is positioned near the end surface 146 of the second bone 140 aligned with the passage 148 in the second bone. A tool (not shown) is used to engage the receiving means 104 in the first end 84 of the second member 80 and drive the second member into the passage 148 of the second bone 120. Since the diameter of the passage 148 is smaller than the diameter φ₃ of the threads 108, advancing the second member 80 into the second bone 120 threads the second member into the passage. The second member 80 is driven into the passage 148 until the entire length L₂ of the second member is within the passage to rigidly secure the second member to the second bone. In this configuration, the end surface 100 of the second member 80 may be substantially aligned with or recessed from the axial end surface 146 of the second bone 140. Where the diameter φ₃ of the threads 108 on the second member 80 varies along the length L₂ of the second member, the variable threads provide additional bone engagement to help rigidly secure the second member to the second bone 140. The second member 80 therefore is not prone to move axially or rotationally relative to the second bone 140 once fully threaded into the passage 148.

The second end 46 of the first member 40 is inserted in the direction A though the passage 134 in the first bone 120 until the threads 66 are positioned adjacent the threads 108 on the second member 80. Since the diameter φ₁ of unthreaded portion 62 and the diameter φ₂ of threaded portion 64 are both smaller than the diameter of the passage 134, the entire shaft 60 of the first member 40 can pass unobstructed through the first bone 120. The surface 54 of the head 50 of the first member 40, however, is larger than the diameter of the passage 134 to prevent the entire first member from passing through the first bone 120. Due to this construction, the first member 120 is therefore free of threaded engagement with the first bone 120 but prevented from passing entirely through the first bone.

Referring to FIG. 4C, subsequent rotation of the first member 40 using the receiving means 52 about the axis 42 in the direction indicated by “R” causes the threads 66 on the shaft 60 to engage the threads 108 in the second passage 106 of the second member 80. The level of threaded engagement between the first and second members 40, 80 controls the level of compression between the first and second bones 120, 140. Continued rotation of the first member 40 in the direction R causes the first member to advance into the second member 80 along the coaxial centerlines 42, 82 in the direction A until the bones 120, 140 abut one another and the head 50 of the first member abuts the end surface 100 of the second member 80. The surface 54 of the head 50 may be contoured to the shape of the end surface 132 of the first bone 120 to ensure substantially uniform contact between the surface and the first bone. Similarly, the proximal end 122 of the first bone 120 and the distal end 144 of the second bone 140 may be shaped, polished, resected, etc., to ensure smooth, uniform engagement between the bones during compression.

Additional rotation of the first member 40 in the direction R applies a compressive force to the first and second bones 120, 140. In particular, as the first member 40 is threaded further into the second member 80, the head 50 of the first member and the threads 108 on the second member draw the bones 120, 140 closer to one another. The variable degree of threaded engagement between the first member 40 and second member 80 allows the level of compression between the bones 120, 140 to be adjusted. More specifically, increased threaded engagement between the first and second members 40, 80 results in increased compressive force between the bones 120, 140. Conversely, decreased threaded engagement between the first and second members 40, 80 results in decreased compressive force between the bones 120, 140. Advantageously, the level of bone 120, 140 compression can therefore be adjusted over time should the need arise to increase or decrease compression levels based on changes in anatomy, treatment, etc.

It will be apparent to one having ordinary skill in the art that the fineness of the threads 66, 108 determines how precise the compression force adjustment can be. Consequently, increasing the fineness of the threads 66, 108 decreases the amount of axial movement of the first member 40 and first bone 120 per revolution of the first member, thereby allowing smaller incremental force adjustments to be made. In any case, once the compressive force level between the first and second bones 120, 140 is set the cooperating threads 66, 108 and threads 90 help to ensure that the first and second members maintain the set level of compressive force between the bones once the device 20 is implanted until/unless adjustments are manually made by the surgeon.

Although the present invention is illustrated as fusing a pair of adjacent bones 120, 140 at a single joint 160, it will be appreciated that the first and/or second members 40, 80 of the fastening device 20 may be configured or adapted to fuse three or more consecutive bones to thereby fuse multiple joints together as shown in FIG. 5. Features in FIG. 5 that are identical to features in FIGS. 1-4C are given the same reference numeral whereas features in FIG. 5 that are similar to features in FIGS. 1-4C are given the suffix “a”. Referring to FIG. 5, the shaft 60a of the first member 40a may have a length L₁ sufficient to extend through both the distal phalanx 120 and the middle phalanx 140 of the hand or foot. In such a case, the second member 80 would be positioned within the proximal phalanx 180 such that securing the first and second members 40, 80 together would rigidly secure all three phalanges 120, 140, 180 together. Accordingly, such a configuration would fuse both the DIP joint 160 and the PIP joint 190.

FIG. 6 illustrates an alternative configuration for a first member 40 in accordance with another aspect of the present invention. In FIG. 6, the head of the first member 40 is omitted. The first end 44 of the first member 40 includes a threaded portion 63 having a series of threads 65. The threads 65 have a variable diameter φ₄ that decreases in a direction extending towards the second end 46 of the first member 40. Alternatively, the diameter φ₄ may be constant along the threaded portion 63 (not shown). In one example, the diameter φ₄ may be about 2.4 mm adjacent the receiving means 52 and decrease to about 1.6 mm at the unthreaded portion 62 of the shaft 60. The diameter φ₄ of the threaded portion 63 is greater than the diameter φ₁ of the unthreaded portion 62 but due to the location of the threaded portion 63 adjacent the receiving means 52 the threaded portion 63 does not prevent or hinder passage of the threaded portion 64 through the first bone 120 and into cooperation with the second member 80. The threaded portion 63 threads into the distal end 130 of the first bone 120 as the first member 40 is threaded into the second member 80 to thereby further help secure the fastening device 20 in place.

FIGS. 7 and 8 illustrate a fastening device 200 in accordance with another aspect of the present invention. The fastening device 200 includes releasably connected first and second members 240, 280 used to secure and compress together the first and second bones 120, 140 forming the joint 160. The first and second members 240, 280 are formed from a durable, biocompatible material, e.g., titanium. The first member 240 has a generally elongated shape and includes a shaft 260 that extends along an axis 242 from a first end 244 to a second end 246, terminating at a pointed tip 266. The first end 244 of the first member 240 includes a head 250 that terminates at an axial end surface 252. A passage 254 extends from the end surface 252 towards the second end 246 of the first member 240. The passage 254 defines receiving means or structure 256 having a hex or hexalobe shape configured to receive a tool, e.g., a hex driver or Allen wrench, to facilitate driving the first member 240 into bone. An outer surface of the head 250 defines a cooperating member 258 having a male Morse taper configuration.

The shaft 260 has a frustoconical cross-section and a length along the axis 242 indicated by L₃. The shaft 250 includes an outer surface 262 on which a series of threads 264 is provided. The threads 260 extend along the entire length L₃ of the first member 240 but may alternatively extend along only a portion or discrete portions of the length (not shown). The threads 264 may be helical, square, etc. The threads 264 have a major or outer diameter φ₅ that decreases in a direction extending towards the second end 246 of the first member 240. The largest thread diameter φ₅ may be wider than the head 250 of the first member 240 or may be narrower than the head. Alternatively, the thread diameter φ₅ may be constant (not shown) along the entire length L₃.

The second member 280 has a generally elongated shape and includes a shaft 300 that extends along an axis 282 from a first end 284 to a second end 288, terminating at a pointed tip 306. The first end 284 of the second member 280 includes a head 290 that terminates at an axial end surface 292. Receiving means or structure 296 is formed on or provided around the head 290 and is configured to receive a tool to facilitate driving the second member 280 into bone. An inner surface of the head 290 defines a passage 294 that extends from the end surface 292 towards the second end 284 of the second member 280. The passage 294 defines a cooperating member 298 that is sized and configured to receive the head 250 of the first member 240. In one example, the cooperating member 298 defined by the passage 294 has a female Morse taper configuration. It will be appreciated, however, that the cooperating members 258, 298 of the first and second members 240, 280 may have any mating or interconnecting configuration that allows the first and second members 240, 280 to connect to one another.

The shaft 300 has a frustoconical cross-section and a length along the axis 282 indicated by L₄. The shaft 300 includes an outer surface 302 on which a series of threads 304 is provided. The threads 340 extend along the entire length L₄ of the second member 280 but may alternatively extend along only a portion or discrete portions of the length (not shown). The threads 304 may be helical, square, or the like. The threads 340 have a major or outer diameter φ₆ that decreases in a direction extending towards the second end 286 of the second member 280. The largest thread diameter φ₆ may be wider than the head 290 of the second member 280 or may be narrower than the head. Alternatively, the thread diameter φ₆ may be constant (not shown) along the entire length L₄.

In use, and referring to FIG. 7, the first bone 120 and second bone 140 are prepared to receive the fastening device 200 similar to how the bones are prepared to receive the fastening device 20. A K-wire and cannulated drill (not shown) are advanced through the first bone 120. The joint 160 may then be reduced or resected, if desired, and the K-wire advanced into the second bone 140. Unlike the process shown in FIGS. 4A-4C, the cannulated drill in FIGS. 7 and 8 forms a passage 136 in the first bone 120 from the end surface 124 at the proximal end 122 towards the distal end 130. The passage 136, however, does not pass entirely through the first bone 120. The passage 136 may be cylindrical or frustoconical but, in any case, the largest diameter of the passage is smaller than the diameter φ₅ of the shaft 260 of the first member 240.

Similarly, the cannulated drill forms a passage 138 in the second bone 140 from the end surface 146 at the distal end 144 towards the proximal end 142 of the second bone. The passage 138, however, does not extend entirely through the second bone 140. The passage 138 may be cylindrical or frustoconical but regardless the largest diameter of the passage is smaller than the diameter φ₆ of the shaft 300 of the second member 280. The passages 136, 138 in the bones 120, 140 are substantially co-axial with one another, i.e., the passages collectively extend in a straight line. Alternatively, the passages 136, 138 may extends at an angle relative to one another (not shown).

The second end 246 of the first member 240 is positioned near the passage 136 in the first bone 120. A tool (not shown) is used to engage the receiving means 256 in the first end 244 of the first member 240 and drive the first member into the passage 136 of the first bone 120. Since the diameter of the passage 136 is smaller than the thread diameter φ₅, advancing the first member 240 into the first bone 120 threads the first member into the passage. The first member 240 is driven into the passage 136 until the entire length L₄ of the shaft 260 is within the passage to rigidly secure the first member into the first bone 120. In this configuration, the transition between the head 250 and the shaft 260 of the first member 240 is substantially aligned with the end surface 124 of the first bone 120. The variable diameter φ₅ of the threads 246 provides additional bone engagement to help rigidly secure the first member 240 to the first bone 120, i.e., the first member is not prone to move axially or rotationally relative to the first bone once fully threaded into the passage 136.

Similarly, the second end 288 of the second member 280 is positioned near the passage 138 in the second bone 140. A tool (not shown) is used to engage the receiving means 296 on the first end 284 of the second member 280 to drive the second member into the passage 138 of the second bone 140. Since the diameter of the passage 138 is smaller than the thread diameter φ₆, advancing the second member 280 into the second bone 140 threads the second member into the passage to rigidly secure the second member to the second bone. The second member 280 is driven into the passage 138 until the entire second member is within the passage. In this configuration, the end surface 292 of the second member 280 may be substantially aligned with or recessed from the end surface 146 of the second bone 140. The variable diameter φ₆ of the threads 304 provides additional bone engagement to help rigidly secure the second member 280 to the second bone 140, i.e., the second member is not prone to move axially or rotationally relative to the second bone once fully threaded into the passage.

When the first and second members 240, 280 are threaded into the respective bones 120, 140 the heads 250, 290 of the first and second members engage one another along the axes 242, 282 to secure the first and second members together. In FIGS. 7 and 8, the cooperating Morse taper configuration of the heads 250, 290 ensures a secure connection between the first and second members 240, 280, thereby securely connecting the bones 120, 140 together and maintaining compression therebetween. In particular, the male Morse taper 258 of the first member 240 mates with the female Morse taper 298 of the second member 280, thereby helping to prevent relative rotational movement between the first and second members.

FIG. 9 illustrates a lateral view of a modified fastening device 220 in which the first member 240 is angled relative to the second member 280 to promote fusion of the first and second bones 120, 140 at a desired angle relative to one another. More specifically, the head 250 of the first member 240 in FIG. 9 is coaxial with the axis 282 of the second member 280 but the shaft 260 extends at an angle, indicated by a, relative to the axis 282, i.e., the axes 242, 282 are angled relative to one another. In one example, the angle α is from about 5° to about 25° promote fusion of the bones 120, 140 in flexion, although it will be appreciated that the angle α may be greater or less and/or may be in one or more different anatomical planes.

FIGS. 10A-10C illustrate various other configurations for the cooperating members that securely fasten the first and second members together, thereby locking the first and second bones 120, 140 together. Features in FIGS. 10A-10C that are identical to features in FIGS. 7 and 8 are given identical reference numerals whereas features in FIGS. 10A-10C that are similar to those in FIGS. 7 and 8 are given the suffix “a”, “b”, and “c”, respectively for each of FIGS. 10A-10C. Referring to FIG. 10A, the first and second members 240a, 280a mate via a Morse taper connection 320 that is reversed compared to the Morse taper connection of FIGS. 7 and 8. In particular, the connecting member 258a of the first member 280a in FIG. 10A constitutes female Morse taper and the connecting member 298a of the second member 280a constitutes a male Morse taper.

In an alternative construction shown in FIG. 10B the heads 250b, 290b of the first and second members 240b, 280b mate via a snap-fit connection 340. More specifically, the cooperating member 258b on the head 240b of the first member 240b constitutes a plurality of circumferentially extending ribs 344 formed on an inner surface 346 of the head. The ribs 344 are U-shaped or V-shaped in the axial direction such that each rib extends circumferentially around the inner surface 346 of the head 240b as well as away from the shaft 300. Alternatively, the ribs 344 may extend perpendicular to the axis 282 around the outer surface (not shown).

The cooperating member 298b on the head 290b of the second member 280b constitutes a cylindrical shape from which a plurality of circumferentially extending ribs 342 project. The ribs 342 are U-shaped or V-shaped in the axial direction such that each rib extends circumferentially around the outer surface 258 as well as towards the shaft 260. Alternatively, the ribs 342 may extend perpendicular to the axis 282 around the outer surface 300 (not shown). In any case, the ribs 342 and the ribs 344 extend in the same direction relative to the direction of the length of the device 220b.

The ribs 342, 344 form a snap-fit connection 340 with one another to secure the first and second members 240b, 280b together. The degree or severity with which the first member 240b is inserted into the second member 280b can be adjusted by varying the number of ribs 342, 344 and/or the size of the ribs engaged with one another. More specifically, an increased radial overlap between cooperating ribs 342, 344, based on the radial thicknesses of the ribs, increases the force necessary to remove the first member 240b from the second member 280b. Likewise, increasing the number of ribs 342 and/or ribs 344 per unit length of the respective first and second member 240b, 280b will increase the force necessary to remove the first member from the second member by increasing the number of ribs engaging one another at any given time. To this end, the head 250b of the first member 240b may have longitudinal slots (not shown) to allow portions of the ribs 342 to move relative to one another to facilitate fastening between the ribs 342, 344.

In yet another construction shown in FIG. 10C, the heads 250c, 290c of the first and second members 240c, 280c mate via modified ribbed connection 360. The connection between the heads 250c, 290c in FIG. 10C is similar to the connection 340 in FIG. 10B, except that the heads in FIG. 10C further include structure to facilitate release of the connection 360. In particular, the head 250c of the first member 240c includes a longitudinally extending, resilient tab 368. The tab 368 is radially movable relative to the remainder of the head 250c and includes a projection 370 extending radially outward from the tab. The projection 370 mates with a corresponding opening 366 in the head 290c of the second member 280c when the ribs 342, 344 engage one another to further secure the first and second members 240c, 280c together. Although FIG. 10C illustrates that no ribs 342 extend around the head 240c in the vicinity of the tab 368 it will be appreciated that ribs may be provided up to the entire length of the head (not shown).

In one example, the projection 370 is positioned on the tab 368 such that the projection snaps into the opening 366 when the head 250c of the first member 240c is fully inserted, i.e., bottoms out, in the head 290c of the second member 280c. Although a single tab 368 and opening 366 are shown it will be appreciated that more tabs and corresponding openings may be provided on the heads 250c, 290c. Furthermore, it will be appreciated that the head 290c may include multiple openings at various positions along its length (not shown) to allow the tab 368 to be locked in different axial positions relative to the head, thereby helping to lock the first and second members 240c, 280c together in different relative axial positions.

Once the heads 250c, 290c are fully connected together, a tool may be inserted into the opening 366 in the head 290c to engage the projection 370 on the head 250 and urge the resilient tab 368 radially inwards. While the tab 368 is in this position, the degree of engagement between the members 240c, 280c can be adjusted by moving the first and second members away from one another, thereby adjusting the degree of compression between the bones 120, 140. Additionally, the projection 370 and the opening 366 allow the first and second members 240c, 280c to be completely disengaged from one another in a non-destructive manner (not shown).

It will be appreciated that, similar to FIG. 9, the first members 240a-c of FIGS. 10A-C may be configured such that the body 260a-c extends at an angle relative to the head 250a-c, respectively. In other words, the axes of the first and second members 240a-c, 280a-c may be angled relative to one another with any head configuration 250a-c and connection 320, 340, 360 illustrated in FIGS. 10A-C in accordance with the present invention.

The present invention is advantageous over prior bone fixation devices. In particular, the fastening device of the present invention allows the surgeon to disconnect the first and second members if clinical or x-ray images indicate that device placement during initial surgery is unsatisfactory. The design also minimizes bone disruption to the distal phalanx and allows the varying degree of compression applied to the bones to be infinite, i.e., dialed or changed to meet clinical needs. In other words, the fastening device of the present invention can be configured for precise compressive force adjustment across a wide range of force levels in accordance with the present invention.

The present invention is also advantageous as it can provide superior fixation in the middle phalanx as the diameter of the second member can be adjusted and/or varied to match the diameter of the bone. On the other hand, in prior bone fixation devices the diameter of the component secured to the middle phalanx was limited in diameter to not larger than the diameter of the smaller component secured to the distal phalanx, thereby undesirably providing lower compressive forces across the DIP joint.

The preferred embodiments of the invention have been illustrated and described in detail. However, the present invention is not to be considered limited to the precise construction disclosed. Various adaptations, modifications and uses of the invention may occur to those skilled in the art to which the invention relates and the intention is to cover hereby all such adaptations, modifications, and uses which fall within the spirit or scope of the appended claims.

Having described the invention, the following is claimed:

1. A fastening device for compressing first and second bones together comprising:
a first member configured to extend entirely through the first bone and including a shaft having a threaded portion and an unthreaded portion, the threaded portion having an outer diameter that is not larger than the diameter of the unthreaded portion; and
a second member having a first threaded portion for securing to the second bone and a second threaded portion for engaging the threaded portion of the first member while the first member extends entirely through the first bone to secure the first and second members together and compress the first and second bones.

2. The fastening device of claim 1, wherein the outer diameter of the threaded portion of the first member is equal to the outer diameter of the unthreaded portion.

3. The fastening device of claim 1, wherein the second member includes a receiving portion for receiving a tool to drive the second member into the second bone.

4. The fastening device of claim 1, wherein the diameter of the first threaded portion of the second member varies along the length of the second member.

5. The fastening device of claim 1, wherein the first threaded portion of the second member extends along the entire length of the second member.

6. The fastening device of claim 1, wherein the first member further includes a head from which the shaft extends.

7. The fastening device of claim 6, wherein the head of the first member includes a tapered surface for engaging an outer surface of the first bone.

8. The fastening device of claim 1, wherein the first bone is a distal phalanx and the second bone is a middle phalanx.

9. The fastening device of claim 1, wherein the first bone is a distal phalanx and the second bone is a proximal phalanx.

10. The fastening device of claim 1, wherein the shaft further includes a second threaded portion having a diameter greater than the diameter of the unthreaded portion for threading into the first bone.

11. A fastening device for compressing first and second bones together comprising:
a first member having a head and a shaft extending from the head, the shaft including threads for engaging the first bone, the head having a first cooperating member; and
a second member having a head and a shaft extending from the head, the head having a second cooperating member that is engageable with the first cooperating member of the first member to compress the first and second bones, the shaft including threads for engaging the second bone.

12. The fastening device of claim 11, wherein the first and second cooperating members form one of a Morse taper connection and a snap-fit connection.

13. The fastening device of claim 11, wherein the first cooperating member includes a plurality of circumferentially extending first ribs and the second cooperating member includes a plurality of circumferentially extending second ribs.

14. The fastening device of claim 13, wherein the first ribs extend towards the shaft of the first member and the second ribs extend away from the shaft of the second member.

15. The fastening device of claim 13, wherein the first cooperating member further includes a radially movable tab that releasably engages an opening in the second cooperating member.

16. The fastening device of claim 11, wherein at least one of the threads of the first member and the threads of the second member are tapered.

17. The fastening device of claim 11, wherein the threads of the first member and the threads of the second member are both tapered.

18. The fastening device of claim 11, wherein the head of the first member includes a receiving portion for receiving a tool to drive the first member into the first bone, the head of the second member including a receiving portion for receiving a tool to drive the second member into the second bone.

19. The fastening device of claim 11, wherein the shaft of the first member extends at an angle relative to the head of the first member.

20. A method of compressing first and second bones together comprising:
providing a first member having a shaft including a threaded portion and an unthreaded portion, the threaded portion having an outer diameter that is not larger than an outer diameter of the unthreaded portion;
providing a second member having a first threaded portion and a second threaded portion;
threading the first threaded portion of the second member into the second bone to secure the second member to the second bone;
forming a passage through the entire first bone having a diameter that is greater than the diameters of the shaft of the first member;
inserting the first member through the passage such that the shaft of the first member extends entirely though the first bone; and
threading the threaded portion of the first member into the second threaded portion of the second member to compress the first and second bones together.

21. The method of claim 20, wherein the first bone is a distal phalanx and the second bone is a middle phalanx.

22. The method of claim 20 further comprising
forming a second passage entirely through a third bone between the first bone and the second bone, the second passage having a diameter that is greater than the diameters of the shaft of the first member; and
inserting the first member through the first and second passages in the first bone and third bone to thread the first member into the second member and compress the first, second, and third bones together.

23. The method of claim 20, wherein the first threaded portion of the second fastener is tapered.

24. The method of claim 19, wherein the outer diameter of the threaded portion of the first member is equal to the outer diameter of the unthreaded portion.

25. The method of claim 20 further comprising inserting a tool into a receiving portion of the second member to drive the second member into the second bone.

26. The method of claim 20, wherein a diameter of the first threaded portion of the second member varies along the length of the second member.

27. The method of claim 20, wherein the first threaded portion of the second member extends along the entire length of the second member.

28. The method of claim 20, wherein the first member includes a head that engages an outer surface of the first bone when the first member is threaded into the second member.

29. The method of claim 28, wherein the head of the first member includes a tapered surface for engaging the outer surface of the first bone.

30. The method of claim 20 further comprising threading a second threaded portion of the shaft into the first bone, the second threaded portion having a diameter greater than the diameter of the unthreaded portion.

31. A method of compressing first and second bones together comprising:
providing a first member having a head and a shaft extending from the head, the head having a first cooperating member, the shaft including threads for engaging the first bone;
providing a second member having a head and a shaft extending from the head, the head having a second cooperating member that is releasably engageable with the first cooperating member of the first member, the shaft including threads for engaging the second bone;
threading the threads of the first member into the first bone to secure the first member to the first bone;
threading the first threaded portion of the second fastener into the second bone to secure the second member to the second bone; and
securing the cooperating members of the first and second members together to compress the first and second bones together.

32. The method of claim 31, wherein the first and second cooperating members form one of a Morse taper connection and a snap-fit connection.

33. The fastening device of claim 31, wherein the first cooperating member includes a plurality of circumferentially extending first ribs and the second cooperating member includes a plurality of circumferentially extending second ribs.

34. The method of claim 31, wherein the first cooperating member further includes a radially movable tab that releasably engages an opening in the second cooperating member.

35. The method of claim 31, wherein the first fastener is threaded into the first bone in a first direction and the second fastener is threaded into the second bone in a second direction opposite the first direction.

36. The method of claim 31, wherein at least one of the threads of the first fastener and the threads of the second fastener is tapered.

37. The method of claim 31, wherein the first bone is a distal phalanx and the second bone is a middle phalanx.

38. The method of claim 31, wherein the first bone is a distal phalanx and the second bone is a proximal phalanx.

39. The method of claim 31, wherein the head of the first member includes a receiving portion for receiving a tool to drive the first member into the first bone, the head of the second member including a receiving portion for receiving a tool to drive the second member into the second bone.

40. The method of claim 31, wherein the shaft of the first member extends at an angle relative to the head of the first member.
